(19) United States
(12) Patent Application Publication
(10) Pub. No.: US 2018/0242896 A1
(43) Pub. Date: Aug. 30, 2018

(54) DEVICES AND METHODS FOR SAMPLE COLLECTION

(71) Applicants: Coyote Bioscience Co., Ltd., Beijing (CN); Coyote Bioscience Yixing Co., Ltd., Wuxi (CN)

(72) Inventors: Xiang Li, Beijing (CN); Xiaobing Mu, Beijing (CN); Yuguang Han, Chengde City (CN); Huiying Feng, Beijing (CN); Yang Zhou, Tongliao City (CN)

(21) Appl. No.: 15/809,839

(22) Filed: Nov. 10, 2017

Related U.S. Application Data
(63) Continuation of application No. PCT/CN2016/083295, filed on May 25, 2016.
Application No. PCT/CN2016/083295 is a continuation-in-part of application No. PCT/CN2015/079706, filed on May 25, 2015.
Application No. PCT/CN2015/079706 is a continuation-in-part of application No. PCT/CN2015/083894, filed on Jul. 13, 2015.
Application No. PCT/CN2015/083894 is a continuation-in-part of application No. PCT/CN2015/085201, filed on Jul. 27, 2015.

(51) Int. Cl. A61B 5/15; B01L 3/02; C12Q 1/6806

(52) U.S. Cl. CPC A61B 5/150755; A61B 5/150022; A61B 5/150213; A61B 5/150236; A61B 5/150244; A61B 2010/0067; A61B 5/150343; A61B 5/150351; B01L 3/022; C12Q 1/6806; A61B 5/150251

(57) ABSTRACT

The present disclosure provides a system for collecting and/or processing a bodily fluid sample of a subject, a method for collecting and/or processing a bodily fluid sample of a subject, and a kit for collecting and/or processing a bodily fluid sample of a subject.

### CROSS-REFERENCE

This application is a continuation of PCT Application Serial No. PCT/CN2016/083295, filed May 25, 2016, which is a continuation-in-part of PCT Application Serial No. PCT/CN2015/079706, filed May 25, 2015, and is a continuation-in-part of PCT Application Serial No. PCT/CN2015/083894, filed Jul. 13, 2015, and is a continuation-in-part of PCT Application Serial No. PCT/CN2015/085201, filed Jul. 27, 2015, which applications are herein incorporated by reference in their entireties for all purposes.

### BACKGROUND

Molecular diagnostics based on polymerase chain reaction (PCR) techniques have been widely used, e.g., in the detection of microorganisms and viruses. Samples (e.g., a blood sample) for use in laboratory testing are often obtained by way of venipuncture or through non-venous puncture, then, the samples are typically transferred to a laboratory for further processing and testing by service providers, e.g., a laboratory technician or a nurse. Samples are normally fractionated (e.g., by centrifugation), purified and processed to extract certain components or molecules therein, which may then be examined to reveal information suitable for diagnosis. The tests often involve amplifying and sequencing of nucleic acid molecules present in the samples, e.g., by PCR (e.g., qPCR).

### SUMMARY

Although there are methods and systems currently available for the collection of bodily fluid or tissue samples from a subject, recognized herein are various limitations associated with such methods and systems. For example, normally, a relatively large quantity of sample may be required to obtain reliable test results, the transportation process can be lengthy, samples can get contaminated or deteriorated in the process, it may take days or even weeks to obtain test results, and it often requires expertise and professional experience to conduct the test and/or interpret test results. Also, during the process of obtaining, transporting and testing of the samples, analyst (e.g., a nurse) can be at the risk of being infected by pathogens or viruses comprised in the sample. These may be problematic for both the subject to be tested and the analyst performing the test. Recognized herein is the need for devices and methods that enable fast, safe, and reliable molecular testing.

The present disclosure provides devices and methods for collecting a sample from a subject in a fast and simple manner, which makes it possible to perform on-site molecular diagnosis quickly and easily, while generating reliable result.

An aspect of the present disclosure provides a system for collecting and/or processing a bodily fluid sample of a subject. The system comprises a collection member comprising (i) at least one collection channel in fluid communication with a first opening at a first end of the collection member, and (ii) a first flange at a second end of the collection member, wherein the first opening permits flow of the bodily fluid sample from a source of the bodily fluid sample to the collection channel. The system may also comprise a collection vessel comprising (i) a container having reagents necessary for nucleic acid amplification, wherein the container has a second opening at an end of the container that permits the collection member to be deposited in the container, and (ii) a second flange that circumscribes the second opening, which second flange engages with the first flange when the collection member is deposited in the container to form a seal.

In some embodiments, when the collection member is deposited in the container through the second opening, the bodily fluid sample flows from the collection channel through the first opening to the container to form a reaction mixture comprising the bodily fluid sample and the reagents.

In some embodiments, the first opening and/or the collection channel is dimensioned to permit flow of the bodily fluid sample via capillary action.

In some embodiments, the collection member further comprises a container in fluid communication with the collection channel, wherein the container has a larger cross-sectional area than the collection channel.

In some embodiments, the collection member further comprises a tip. The tip may include a finger prick. In some embodiments, the tip is a radially extending tip that is unsymmetrical with respect to longitudinal axis of the collection member.

In some embodiments, the collection member comprises a third opening that is dimensioned to seal the collection channel upon application of the first flange against an external object. In some embodiments, the first flange circumscribes the third opening. The external object may be a finger or a plug (e.g., a rubber plug).

In some embodiments, the collection member is removably stored in a first housing and the collection vessel is removably stored in a second housing. The second housing may be attached to the first housing. The attachment between the first housing and the second housing may be removable.

In some embodiments, the collection vessel includes a polymeric membrane adjacent to the second flange that is penetrable by the collection member. The polymeric membrane may be sealable or resealable. In some embodiments, the polymeric membrane is made from parafilm.

In some embodiments, the bodily fluid sample is a blood sample. The blood sample may be a whole blood sample.

In some embodiments, the collection channel and/or the container is substantially free of an anticoagulant.

In some embodiments, the reagents necessary for nucleic acid amplification include one or more primers and a polymerizing enzyme. The one or more primers may have sequences that are selected to assay for a presence of a disease in the subject. The disease may be an infectious disease or cancer. In some embodiments, the reagents include Mg or Mn ions.

In some embodiments, the collection vessel is adapted to stably store the mixture for a time period of at least about 5 minutes.

In some embodiments, the bodily fluid sample has a volume that is less than about 1 mL.

In some embodiments, the source of the bodily fluid sample is a pool of the bodily fluid sample in a storage vessel. In some embodiments, the source is a tissue of the subject that is accessible through a puncture in the tissue.

In some embodiments, the system further comprises identifying information of the subject. The identifying information may be on the collection vessel, the collection member, or a housing of the collection vessel or the collection member. The identifying information may be anonymous. In some embodiments, the identifying information is on a barcode. In some embodiments, the identifying information is in a radio-frequency identification (RFID) tag.

In some embodiments, the system further comprises a heating member adjacent to the collection vessel, wherein the heating member heats the reaction mixture during nucleic acid amplification. In some embodiments, the heating member is a thermal cycler that subjects the reaction mixture to one or more heating and cooling cycles during the nucleic acid amplification.

In some embodiments, the heating member comprises a receptacle that is dimensioned to accept the collection vessel.

In some embodiments, the collection member comprises a plurality of collection channels.

In another aspect, the present disclosure provides a method for collecting and/or processing a bodily fluid sample of a subject. The method comprises: providing a collection member comprising (i) at least one collection channel in fluid communication with a first opening at a first end of the collection member, and (ii) a first flange at a second end of the collection member; positioning the first opening of the collection member adjacent to a source of the bodily fluid sample such that the bodily fluid sample flows from the source through the first opening to the collection channel; depositing the collection member in a collection vessel comprising (i) a container having reagents necessary for nucleic acid amplification, and (ii) a second flange that circumscribes the second opening, wherein upon depositing the collection member in the collection vessel, the second flange engages with the first flange to form a seal; and flowing the bodily fluid sample from the collection channel through the first opening to the container to form a reaction mixture comprising the bodily fluid sample and the reagents.

In some embodiments, the collection vessel includes a polymeric membrane adjacent to the second flange that is penetrable by the collection member.

In some embodiments, the depositing comprises penetrating the polymeric membrane with the collection member.

In some embodiments, the bodily fluid sample is a blood sample.

In some embodiments, the collection channel and/or the container is substantially free of an anticoagulant.

In some embodiments, the reagents necessary for nucleic acid amplification include one or more primers and a polymerizing enzyme. The one or more primers may have sequences that are selected to assay for a presence of an infectious disease in the subject.

In some embodiments, the bodily fluid sample has a volume that is less than about 1 mL.

In some embodiments, the method further comprises providing identifying information of the subject.

In some embodiments, the method further comprises disposing the collection vessel adjacent to a heating member.

In some embodiments, the method further comprises heating the reaction mixture with the heating member. In some embodiments, the heating comprises subjecting the reaction mixture to one or more heating and cooling cycles.

In some embodiments, when the collection member is deposited in the collection vessel, the first opening is submerged in the reagents.

In some embodiments, the method further comprises performing nucleic acid amplification using reaction mixture in the collection vessel.

In some embodiments, (b)-(d) of the method are performed in a time period of less than about 10 minutes. In some embodiments, the time period is less than about 1 minute. In some embodiments, the time period is less than about 30 seconds.

In some embodiments, the bodily fluid sample has a volume that is less than about 1 mL.

In some embodiments, the flowing in (d) comprises subjecting the bodily fluid sample to positive pressure.

In another aspect, the present disclosure provides a kit for collecting and/or processing a bodily fluid sample of a subject. The kit may comprise: a collection member comprising (i) at least one collection channel in fluid communication with a first opening at a first end of the collection member, and (ii) a first flange at a second end of the collection member, wherein the first opening permits flow of the bodily fluid sample from a source of the bodily fluid sample to the collection channel; and a collection vessel comprising (i) a container having reagents necessary for nucleic acid amplification, wherein the container has a second opening at an end of the container that permits the collection member to be deposited in the container, and (ii) a second flange that circumscribes the second opening, which second flange engages with the first flange when the collection member is deposited in the to form a seal. When the collection member is deposited in the container through the second opening, the bodily fluid sample may flow from the collection channel through the first opening to the container to form a reaction mixture comprising the bodily fluid sample and the reagents. The kit may also comprise instructions that permit a user to use the collection member to (i) collect the bodily fluid sample from the source, and (ii) deposit the bodily fluid sample into the collection vessel to provide the reaction mixture.

The kit may further comprise a first housing and a second housing attached to the first housing, wherein the collection member is removably stored in the first housing and the collection vessel is removably stored in the second housing. The attachment between the first housing and the second housing may be removable.

In some embodiments, the bodily fluid sample is a blood sample.

In some embodiments, the collection channel and/or the container is substantially free of an anticoagulant.

In some embodiments, the reagents necessary for nucleic acid amplification include one or more primers and a polymerizing enzyme. In some embodiments, the one or more primers have sequences that are selected to assay for a presence of a disease in the subject. The disease may be an infectious disease or cancer.

In some embodiments, the kit further comprises identifying information of the subject. In some embodiments, the identifying information is on the collection vessel, the collection member, or a housing of the collection vessel or the collection member. The identifying information may be anonymous. In some embodiments, the identifying information is on a barcode. In some embodiments, the identifying information is in a radio-frequency identification (RFID) tag.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1D demonstrate a workflow for sample collection using a sample collection member of the present disclosure;

FIG. 2 illustrates a collection vessel of the present disclosure;

FIG. 3 illustrates a system of the present disclosure;

FIG. 4 illustrates another system of the present disclosure. FIG. 4B demonstrates the system with caps of housing removed, and FIG. 4C shows a stereo view of the system with the caps of the housings removed;

FIG. 5 illustrates a system of the present disclosure;

FIG. 6 shows enlarged views of part of the system of the present disclosure; and

FIG. 7 shows sectional view and stereo view of the system after assembling.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “substantial”, as used herein, generally refers to more than a minimal or insignificant amount; and “substantially” generally refers to more than minimally or insignificantly. The term “substantially free of,” as used herein with respect to an amount, quantity or concentration of a substance, generally means that that there is less than about 10% (v/v), less than about 5% (v/v), less than about 4% (v/v), less than about 3% (v/v), less than about 2% (v/v), less than about 1% (v/v), less than about 0.1% (v/v), less than about 0.01% (v/v), less than about 0.001% (v/v), or less than about 0.0001% (v/v) of the substance in a mixture or a device, or a component of the device.

The term “sample,” as used herein, generally refers to a tissue or a bodily fluid sample. For example, a sample can be but is not limited to a blood sample, or a portion thereof. A sample may contain or be suspected of containing a nucleic acid molecule. The sample can include cellular material. The sample can include nucleic acid material, such as deoxyribonucleic acid (DNA) or ribonucleic acid (RNA). For example, a subject sample can be a biological sample containing one or more nucleic acid molecules. The biological sample can be obtained or obtainable (e.g., extracted or isolated) from a bodily sample of a subject that can be selected from blood (e.g., whole blood), plasma, serum, urine, saliva, mucosal excretions, sputum, stool and tears. The bodily sample can be a fluid or tissue sample (e.g., skin sample) of the subject. In some examples, the sample is obtained from a cell-free bodily fluid of the subject, e.g., whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

A sample may be of any suitable size or volume. In some examples, a small volume comprises no more than about 5 mL; no more than about 4 mL; no more than about 3 mL; no more than about 2 mL; no more than about 1 mL; no more than about 500 μL; no more than about 250 μL; no more than about 100 μL; no more than about 75 μL; no more than about 50 μL; no more than about 35 μL; no more than about 25 μL; no more than about 20 μL; no more than about 15 μL; no more than about 10 μL; no more than about 8 μL; no more than about 6 μL; no more than about 5 μL; no more than about 4 μL; no more than about 3 μL; no more than about 2 μL; no more than about 1 μL; no more than about 0.8 μL; no more than about 0.5 μL; no more than about 0.3 μL; no more than about 0.2 μL; no more than about 0.1 μL; no more than about 0.05 μL; or no more than about 0.01 μL.

The term “point of care,” as used herein, generally refers to locations where a subject may be cared for (e.g., by testing, monitoring, treatment, diagnosis, guidance, sample collection, identification (ID) verification, medical services, non-medical services, etc.), and may include but not limited to, a subject's home, a subject's business, the location of a healthcare provider (e.g., doctor), hospitals, emergency rooms, operating rooms, clinics, health care professionals' offices, laboratories, retailers—e.g., pharmacies (e.g., retail pharmacy, clinical pharmacy, hospital pharmacy), drugstores, supermarkets, grocers, etc.—transportation vehicles (e.g., car, boat, truck, bus, airplane, motorcycle, ambulance, mobile unit, fire engine/truck, emergency vehicle, law enforcement vehicle, police car, or other vehicle configured to transport a subject from one point to another, etc.), traveling medical care units, mobile units, schools, day-care centers, security screening locations, combat locations, health assisted living residences, government offices, office buildings, tents, sample acquisition sites (e.g., blood collection centers), or any other point of care location described elsewhere in the present application.

The term “bodily fluid”, as used herein, generally refers to any fluid obtainable from a subject. A bodily fluid may include but not limited to, e.g., blood, urine, saliva, tears, sweat, a bodily secretion, a bodily excretion, or any other fluid originating in or obtainable from a subject. In particular, bodily fluids include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions.

As used herein, a “collection member” can be disposable, e.g., it can be used once and disposed. A collection member may also comprise one or more disposable components, wherein each of said components may be used once and disposed. Alternatively or in addition, a collection member may be reusable or may comprise one or more reusable components, which for example may be reused any number of times.

As used herein, a “collection channel” may be capable of receiving one or more types of sample. For example, collection channel may be capable of receiving two different types of bodily fluid sample (e.g., blood, tears).

The term “needle”, as used herein, generally refers to any article capable of penetrating a tissue or tissue surface of a subject, thereby introducing material into or removing material from the said tissue. In some embodiments, a needle can be a sharp-pointed slender instrument.

The term “button”, as used herein, generally refers to a mechanical component that can be compressed or depressed to various positions and/or levels. A button can be depressable and extendable. A button can be of any form or shape suitable for being compressed or depressed to various positions and/or levels, e.g., cylindrical, cubical, bar-shaped, rod-shaped, etc. A button of the present disclosure can be configured to drive the movement of a collection channel and/or a collection member. A button can also be configured to initiate or drive the movement of a sample into or out of a collection channel.

The term “kit”, as used herein, generally refers to a combination of two or more components, wherein said two or more components can be comprised in a single package or container. Alternatively, said two or more components can be separately comprised in two or more independent packages or containers.

The term “membrane,” as used herein, generally refers to a structure that separates at least two volumes, or that separates a volume from the external environment. A membrane may be a synthetic membrane, e.g., a membrane formed of a solid state material (e.g., semiconductor, metal, semi-metal or non-metal) or polymeric material (e.g., a polymeric membrane). For example, a membrane can be formed by an opaque, transparent, or translucent material sealing a collection vessel and separating it from the external environment. In some embodiments, the membrane can be a polymeric membrane made from parafilm.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof including but not limited to peptide nucleic acid (PNA). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded. A nucleic acid may comprise one or more modified nucleotides, e.g., methylated nucleotides and nucleotide analogs.

The term “polymerase,” as used herein, generally refers to any enzyme capable of catalyzing a polymerization reaction. Examples of polymerases include, without limitation, a nucleic acid polymerase, a transcriptase or a ligase. A polymerase can be a polymerization enzyme or a polymerizing enzyme.

The term “subject,” as used herein, generally refers to an animal or other organism, e.g., a mammalian species (e.g., human), avian (e.g., bird) species, or plant. Mammals include, but are not limited to, murines, simians, humans, farm animals, sport animals, and pets. A subject can be an individual that has or is suspected of having a disease or a pre-disposition to the disease, or an individual that is in need of therapy or suspected of needing therapy. A subject can be a patient.

As used herein, the term “anticoagulant” is an agent capable of maintaining a sample (e.g., a blood sample) in liquid form. An anticoagulant can be an anti-coagulating agent, such as, for example, heparin (e.g., lithium heparin or sodium heparin) or ethylene diamine tetra acetic acid (EDTA), in some cases integrated with external peripherals for integrated tests or services.

The present disclosure provides devices, methods and systems for obtaining, processing and analyzing a sample. Various aspects of the devices, systems and methods described herein may be applied to any of the particular devices, systems and methods set forth below. Devices, systems and methods provided herein may be applied as a standalone device, system or method, or as part of an integrated system, e.g., in a system involving point of care services.

System for Collecting and/or Processing a Bodily Fluid Sample of a Subject

An aspect of the present disclosure provides a system for collecting and/or processing a sample (e.g., a bodily fluid sample, e.g., a blood sample) of a subject. The system may comprise a collection member and a collection vessel. The collection member may comprise (i) at least one collection channel in fluid communication with a first opening at a first end of the collection member, and (ii) a first flange at a second end of the collection member. The first opening may permit flow of the sample (e.g., a bodily fluid sample, such as a blood sample) from a source of the sample to the collection channel. The collection vessel may comprise (i) a container having reagents necessary for nucleic acid amplification, the container may have a second opening at an end of the container that permits the collection member to be deposited in the container. The collection vessel may further comprise (ii) a second flange that circumscribes the second opening. The second flange may engage with the first flange when the collection member is deposited in the container to form a seal. In some embodiments, the collection member comprises a plurality of collection channels.

The collection member can include at least one or a plurality of collection channels. Each collection channel can be in fluid communication with an opening to collect the bodily fluid sample. In some cases, multiple channels are in fluid communication with an opening.

When the collection member is deposited in the container through the second opening, the bodily fluid sample may flow from the collection channel through the first opening to the container to form a reaction mixture comprising the bodily fluid sample and the reagents. The opening (e.g., the first opening) and/or the collection channel may be dimensioned to permit flow of the bodily fluid sample via capillary action. For example, the opening (e.g., the first opening) and/or the collection channel may have a diameter of about 0.1 mm or less, 0.2 mm or less, 0.3 mm or less, 0.4 mm or less, 0.5 mm or less, 0.6 mm or less, 0.7 mm or less, 0.8 mm or less, 0.9 mm or less, 1.0 mm or less, 1.1 mm or less, 1.2 mm or less, 1.3 mm or less, 1.4 mm or less, 1.5 mm or less, 1.6 mm or less, 1.7 mm or less, 1.8 mm or less, 1.9 mm or less, 2.0 mm or less, 2.5 mm or less, 3.0 mm or less, 3.5 mm or less. In some cases, the collection channel may have a length of about, 0.3 mm or less, 0.4 mm or less, 0.5 mm or less, 0.6 mm or less, 0.7 mm or less, 0.8 mm or less, 0.9 mm or less, 1.0 mm or less, 1.1 mm or less, 1.2 mm or less, 1.3 mm or less, 1.4 mm or less, 1.5 mm or less, 1.6 mm or less, 1.7 mm or less, 1.8 mm or less, 1.9 mm or less, 2.0 mm or less, 2.5 mm or less, 3.0 mm or less, 3.5 mm or less, 3.5 mm or less, 4.0 mm or less, 4.5 mm or less, 5.0 mm or less, 5.5 mm or less, 6.0 mm or less, 6.5 mm or less, 7.0 mm or less, 7.5 mm or less, 8.0 mm or less, 8.5 mm or less, 9.0 mm or less, 9.5 mm or less, 10.0 mm or less, 10.5 mm or less, 11.0 mm or less, 11.5 mm or less, 12.0 mm or less, 12.5 mm or less, 13.0 mm or less, 13.5 mm or less, 14.0 mm or less, 15.0 mm or less, 16.0 mm or less, 17.0 mm or less, 18.0 mm or less, 19.0 mm or less, or 20.0 mm or less.

The collection member may further comprise a container in fluid communication with the collection channel. The container may be used to collect the bodily fluid sample from the collection channel. The container may have a larger cross-sectional area than the collection channel.

In some embodiments, the collection member may also comprise a tip. The tip may include a finger prick. In some embodiments, the tip is a radially extending tip that is unsymmetrical with respect to the longitudinal axis of the collection member, and a pointed end may be formed thereby. In some cases, the collection member may comprise a third opening, which may be dimensioned to seal the collection channel upon application of the first flange against an external object. The first flange may circumscribe the third opening. The external object may be a finger of a user or a plug, such as a rubber plug, or a plug made of other suitable materials (e.g., paper, polymeric materials, etc.). In some cases, a user places a finger on the third opening to retain the bodily fluid sample in the collection member (e.g., via negative pressure).

In some embodiments, the collection member can further include a cover positioned adjacent to the first end of the collection member, wherein the cover is configured to prevent the opening and/or the tip (e.g., the finger prick, when present) from being exposed to the environment and to keep them clean. The cover may be formed from an opaque, transparent, or translucent material. The cover may also be formed from a material penetrable with a collection member.

The collection member may be removably stored in a first housing and the collection vessel may be removably stored in a second housing. The second housing may be attached to the first housing. The attachment between the first housing and the second housing may be removable. For example, the first housing and the second housing may be attached to each other with a rubber band, a clip, a hook, or any other device capable of keeping two or more components of a system temporarily and/or permanently together.

In some embodiments, the collection vessel may include a membrane (e.g., a polymeric membrane, such as a parafilm) adjacent to the second flange of the collection vessel. The membrane may seal the contents inside the collection vessel to separate it from the surrounding environment. The membrane may be penetrable (e.g., by the collection member). For example, the collection member may pierce the membrane. As an alternative, the membrane may include at least one slit (e.g., single slit or crossing slits), which may enable the membrane to be penetrated by the collection and sealed when the collection membrane is removed.

The membrane may be sealable or resealable. In some embodiments, the membrane is made from parafilm. The membrane may be a synthetic membrane, e.g., a membrane formed of a solid state material (e.g., semiconductor, metal, semi-metal or non-metal) or polymeric material. For example, a membrane can be formed by an opaque, transparent, or translucent material sealing a collection vessel and separating it from the external environment.

The sample can be a tissue or a bodily fluid sample, or a fraction thereof. In some cases, the sample is a “bodily fluid” sample, which may include but not limited to blood, urine, saliva, tears, sweat, a bodily secretion, a bodily excretion, or any other fluid originating in or obtainable from a subject. In particular, the sample may include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions. For example, a sample can be a blood sample, or a portion thereof, which may include but not limited to a whole blood sample, a sample comprising red blood cells, plasma sample, serum sample, buffy coat sample, a sample comprising white blood cells, etc. The blood sample can be obtained directly from the subject, e.g., the sample can be analyzed or tested (e.g., by amplification or sequencing) without further processing (e.g., by centrifugation, purification, etc.)

The collection channel and/or the container may be substantially free of an anticoagulant.

The reagents necessary for nucleic acid amplification may include one or more primers and a polymerizing enzyme. The reagents may further include one or more of the following: primer(s), probe(s), nucleotides (e.g., nucleotide triphosphates containing deoxyribose, or dNTP), polymerizing enzyme (or polymerase), reverse transcription enzyme (or reverse transcriptase), and/or amplification buffer. The reagents can include any one, two, three, four, five, or all of the primer(s), probe(s), nucleotides, polymerizing enzyme, reverse transcription enzyme and amplification buffer. In some embodiments, the reagents include Mg or Mn ions.

The one or more primers may have sequences that are selected to assay for a presence of a disease in the subject. The disease may be an infectious disease or cancer. In some embodiments, the disease may be associated with a virus e.g., an RNA virus or a DNA virus. For example, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus can be selected from the group consisting of H1N1 virus, H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease may be associated with a pathogenic bacterium (e.g., Mycobacterium tuberculosis) or a pathogenic protozoan (e.g., Plasmodium).

The collection vessel may be adapted to stably store the mixture for a time period of at least about 5 minutes. In some embodiments, the collection vessel may be adapted to stably store the mixture for a time period of at least about 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, or 1 month.

The sample may be a bodily fluid sample having a volume of no more than about 5 mL; no more than about 4 mL; no more than about 3 mL; no more than about 2 mL; no more than about 1 mL; no more than about 500 μL; no more than about 250 μL; no more than about 100 μL; no more than about 75 μL; no more than about 50 μL; no more than about 35 μL; no more than about 25 μL; no more than about 20 μL; no more than about 15 μL; no more than about 10 μL; no more than about 8 μL; no more than about 6 μL; no more than about 5 μL; no more than about 4 μL; no more than about 3 μL; no more than about 2 μL; no more than about 1 μL; no more than about 0.8 μL; no more than about 0.5 μL; no more than about 0.3 μL; no more than about 0.2 μL; no more than about 0.1 μL; no more than about 0.05 μL; or no more than about 0.01 μL. For example, the sample can have a volume of about 0.01 μL to about 5 mL, about 0.01 μL to about 4mL, about 0.01 μL to about 3mL, about 0.01 μL to about 2mL, about 0.01 μL to about 1 mL, about 0.01 μL to about 0.5 μL, about 0.01 μL to about 0.4 μL, about 0.01 μL to about 0.3 μL, about 0.01 μL to about 0.2 μL, about 0.01 μL to about 0.1 μL, about 0.01 μL to about 0.05 μL.

In some embodiments, the source of the bodily fluid sample is a pool of the bodily fluid sample in a storage vessel. In some embodiments, the source is a tissue of the subject that is accessible through a puncture in the tissue.

In some embodiments, the system may further comprise identifying information of the subject. The identifying information may be on the collection vessel, the collection member, or a housing of the collection vessel or the collection member. The identifying information may be anonymous. In some embodiments, the identifying information is on a barcode. In some embodiments, the identifying information is in a radio-frequency identification (RFID) tag.

The system of the present disclosure may further comprise a heating member adjacent to the collection vessel, wherein the heating member heats the reaction mixture during nucleic acid amplification. In some embodiments, the heating member is a thermal cycler that subjects the reaction mixture to one or more heating and cooling cycles during the nucleic acid amplification. The heating member may comprise a receptacle that is dimensioned to accept the collection vessel.

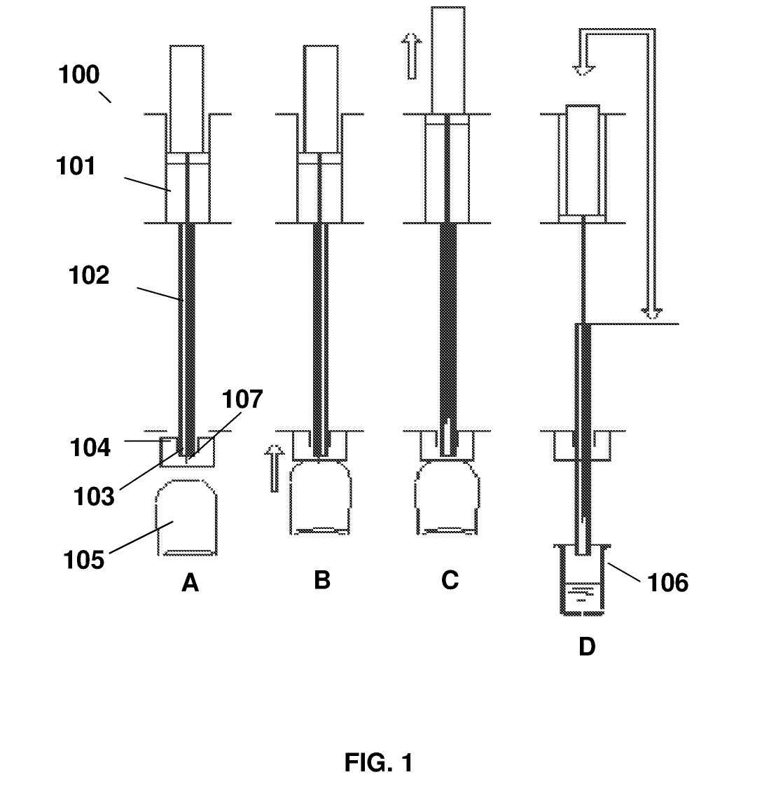

With reference to FIGS. 1A-1D, a collection member 100 is shown. The collection member 100 may include a first flange 101, at least one collection channel 102 having a first opening 103 at an end thereof. The collection member may also comprise a finger prick 107 at or adjacent to the end having the opening 103. In certain cases, the collection channel may be substantially free of any anticoagulant.

In some instances, a cover 104 may be provided. The cover 104 may be positioned at the end of the collection channel comprising the opening 103, wherein the cover is configured to prevent the opening 103 and the finger prick 107 from being exposed to the environment and to keep them clean. For example, when the sample is blood sample, the cover may be configured in an appropriate shape and position capable of protecting the opening and the finger prick, keeping them clean, and covering up the bloody tip of the finger prick after sampling. In one non-limiting example, the cover 104 may be fitted over a portion of the collection channel 102 or be fitted over the finger prick 107. The cover 104 may be detachable from the collection channel 102. In some instances, the cover 104 may be completely separable from the collection channel 102, or may retain a portion that is connected to the collection channel 102, e.g., but not limited to being hinged or otherwise linked to the collection channel. The cover 104 may cover a portion of the collection channel 102 containing an opening at an end thereof. The cover 104 may prevent material, e.g., air, fluid, or particulates, from entering the channels, when the cover is in place. The cover 104 may attach to the collection channel 102 using any technique known or later developed in the art. For instance, the cover may be snap fit, twist on, friction-fit, clamp on, have magnetic portions, tie in, utilize elastic portions, and/or may removably connect to the collection channel. The cover may also be directly or indirectly connected to the collection channel, e.g., through an intermediate material positioned between the collection channel and the cover. The cover may form a fluid-tight seal with the collection channel. The cover may be formed from an opaque, transparent, or translucent material. The cover may be formed from a material penetrable with a collection channel and/or a finger prick.

The collection channel may be any form of pathways able to transport and store (at least transiently) a sample, such a bodily fluid sample, e.g., blood. The collection channel may have any shape or size, some embodiments are configured such that the channel exhibits a capillary action when in contact with sample fluid. In some instances, the channel may have a cross-sectional area of less than or equal to about 10 mm², 7 mm², 5 mm², 4 mm², 3 mm², 2.5 mm², 2 mm², 1.5 mm², 1 mm², 0.8 mm², 0.5 mm², 0.3 mm², or 0.1 mm². The cross-sectional size may remain the same or may vary along the length. Some channels are straight in configuration. Some embodiments may have curved or other shaped path shapes alone or in combination with straight portions. Some may have different orientations relative to the first flange 101. For example, when the collection member 100 is held substantially horizontally, one or more channels may slope downward, slope upward, or not slope at all as it carries fluid away from the initial collection point on the collection member.

The finger prick 107 may be actuated to extend away from the collection channel for collection of a sample from the source thereof. The finger prick 107 may also be configured to retract into the collection channel upon collection of the sample from the source thereof

It should be understood that although the first flange 101, the collection channel 102, the finger prick 107, and the cover 104 are recited as separate parts, one or more of those parts may be integrally formed to simplify manufacturing and such integration is not excluded herein.

FIG. 1A shows that a subject aligns his/her finger 105 with the cover 104. As demonstrated in FIG. 1A, the finger prick 107 can be actuated to extend away from the collection channel 102 for collection of a sample. FIG. 1B shows that the finger moves towards the finger prick, thereby also pressing the cover towards the finger prick. As a result, the finger prick (e.g., comprising a needle) may penetrate through the cover and may be exposed. Then, the exposed finger prick may contact with and prick the finger, thereby permitting the sample (e.g., blood) to flow out of the finger. FIG. 1C shows that the finger prick may then retract into the collection channel, which can be achieved by, e.g., a pressure created by the finger while pressing against the finger prick, thereby the sample may enter into the collection channel (e.g., a capillary) through the first opening 103 at the end of the collection channel. FIG. 1D shows that when sufficient sample has been collected in the collection channel, a positive pressure may be applied to the collection member, which in turn may actuate a portion of the collection channel covered by the cover to protrude out of the cover. If desired, a further positive pressure may be applied to the collection member, thereby causing the sample collected to flow from the collection channel to the opening 103.

The sample may flow into a collection vessel 106, which may be capable of being in fluid communication with the collection channel. The collection vessel may comprise reagents necessary for nucleic acid amplification. Thus, the sample from the collection channel and reagents comprised in the collection vessel may form a reaction mixture. In some cases, the collection vessel may be substantially free of any anticoagulant. The reagents necessary for nucleic acid amplification may include one or more primers (e.g., primers specific for amplifying certain target nucleic acids), it may also include a polymerizing enzyme. The reagents may further include Mg or Mn ions. The reagents may also include one or more of the following: primer(s), probe(s), nucleotides (e.g., nucleotide triphosphates containing deoxyribose, or dNTP), polymerizing enzyme (or polymerase), reverse transcription enzyme (or reverse transcriptase), and/or amplification buffer. The reagents can include any one, two, three, four, five, or all of the primer(s), probe(s), nucleotides, polymerizing enzyme, reverse transcription enzyme and amplification buffer. The primers may have sequences selected to assay for a presence of an infectious disease in the subject. For example, the primers may have sequences selected to assay for the presence and/or amount of one or more pathogens, including but not limited to H1N1 virus, H3N2 virus, H7N9 virus and H5N1 virus, adenovirus type 55 (ADV55) or adenovirus type 7 (ADV7, armored RNA-hepatitis C virus (RNA-HCV), a pathogenic bacterium (e.g., Mycobacterium tuberculosis) or a pathogenic protozoan (e.g., Plasmodium).

The reaction mixture can include reagents necessary to complete nucleic acid amplification (e.g., DNA amplification, RNA amplification), with non-limiting examples of such reagents including primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents. The reaction mixture can also include an enzyme that is suitable to facilitate nucleic acid amplification, e.g., a polymerizing enzyme (also “polymerase” herein). The polymerase can be a DNA polymerase for amplifying DNA. Any suitable DNA polymerase may be used, including commercially available DNA polymerases. The DNA polymerase can be capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases.

In some cases, a DNA sample can be generated from an RNA sample. This can be achieved using reverse transcriptase, which can include an enzyme that is capable of incorporating nucleotides to a strand of DNA, when bound to an RNA template. Any suitable reverse transcriptase may be used. Non-limiting examples of reverse transcriptases include HIV-1 reverse transcriptase, M-MLV reverse transcriptase, AMV reverse transcriptase, telomerase reverse transcriptase, and variants, modified products and derivatives thereof

Nucleic acid amplification reaction can include one or more primer extension reactions to generate amplified product(s). In PCR, for example, a primer extension reaction can include a cycle of incubating a reaction mixture at a denaturation temperature for a denaturation duration and incubating a reaction mixture at an elongation temperature for an elongation duration. Denaturation temperatures may vary depending upon, e.g., the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be at least about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

As an alternative, in isothermal amplification, the temperature can be fixed (i.e., maintained constant and not cycled), and amplification product(s) can be generated using a primer set and a polymerase with high strand displacement activity in addition to a replication activity. An example of a polymerase that may be suitable for use in isothermal amplification is Bst polymerase. The temperature can be fixed between about 50° C. and 80° C., or 60° C. and 65° C. In loop mediated isothermal amplification (LAMP), e.g., a template nucleic acid molecule can be amplified using a polymerase and a primer set having at least 2, 3, 4, or 5 primers.

The amplification of the template nucleic acid molecule and detection of the target nucleic acid molecule can be performed in the same system, e.g., a vessel. In some cases, the system is a tube that is configured for nucleic acid amplification, e.g., an eppendorf PCR tube.

The collection vessel 106 may include identifying information of the subject. The identifying information may be anonymous, it may also be on a barcode or in a radio-frequency identification (RFID) tag. The barcode can include a string of characters, e.g., letters and/or numbers.

In some embodiments, the collection vessel 106 may be sealed with a membrane (e.g., a polymeric membrane) before being in fluid communication with the collection channel. The membrane may be a synthetic membrane, e.g., a membrane formed of a solid state material (e.g., semiconductor, metal, semi-metal or non-metal) or polymeric material. For example, a membrane can be formed by an opaque, transparent, or translucent material (e.g., a parafilm) sealing a collection vessel and separating it from the external environment. Thus, when sufficient sample has been collected in the collection channel, a positive pressure may be applied to the collection member, which in turn may actuate a portion of the collection channel covered by the cover to protrude out of the cover, meanwhile, the finger prick 107 may be simultaneously actuated to extend away from the collection channel and be exposed. Then, upon a further positive pressure applied to the collection member, the finger prick may be actuated to penetrate the membrane sealing the collection vessel, rendering it in fluid communication with the collection channel, thereby permitting the sample collected to flow from the collection channel to the collection vessel.

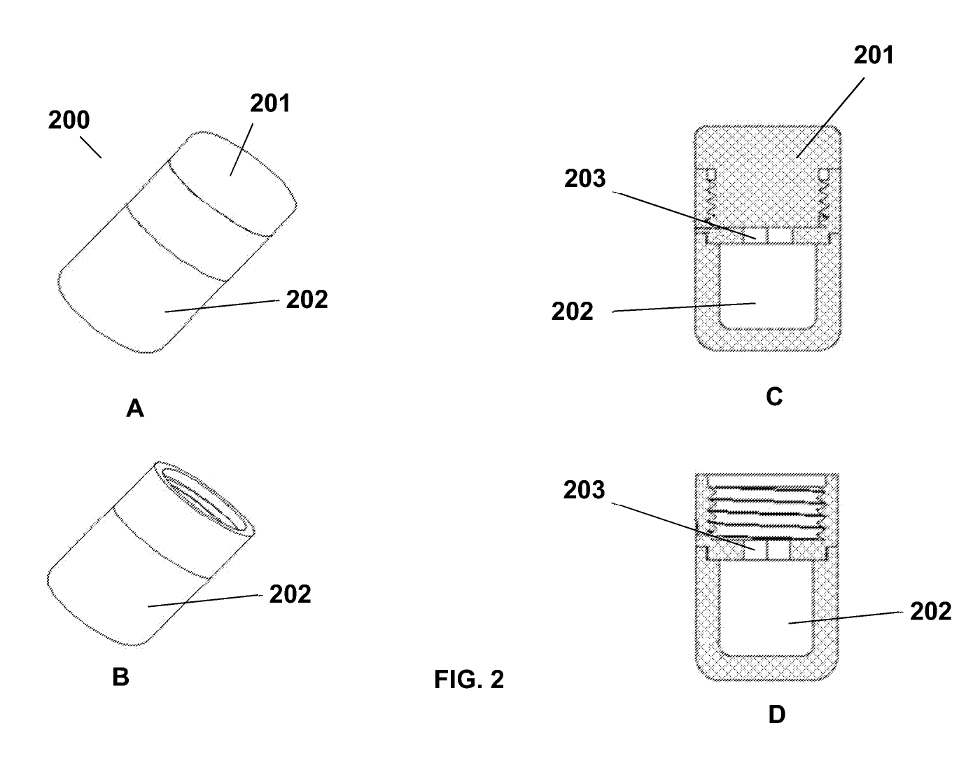

FIG. 2A provides a non-limiting example of a collection vessel 200, comprising a vessel cap 201 and a vessel body 202. FIG. 2B demonstrates a vessel body 202 without a cap. FIG. 2C shows a perspective view of the collection vessel 200, wherein the vessel body 202 comprises a polymeric membrane 203 to separate the contents (e.g., reagents necessary for nucleic acid amplification) therein from the external environment. FIG. 2D shows a perspective view of the vessel body 202 without a cap.

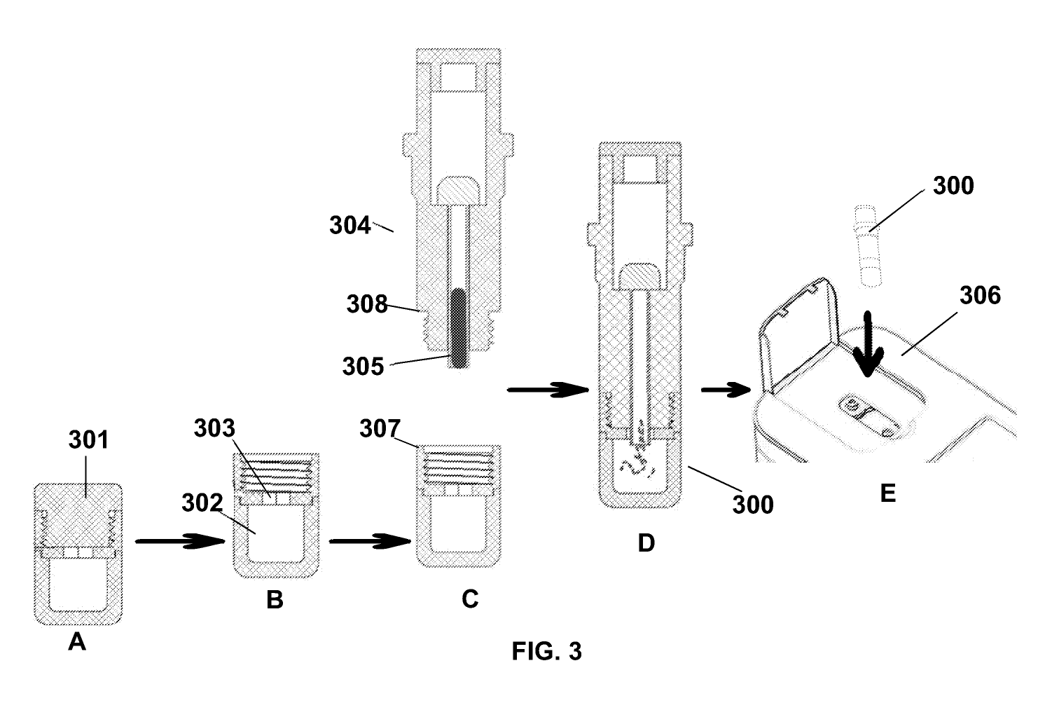

FIG. 3 shows use of a system 300 according to one non-limiting example of the present disclosure for nucleic acid amplification. The system 300 comprises a collection member 304 and a collection vessel, wherein the collection vessel comprises a cap 301 and a vessel body 302 containing reagents necessary for nucleic acid amplification. The vessel body 302 further comprises a membrane 303 sealing the contents (e.g., reagents necessary for nucleic acid amplification) from the environment. Before analysis, the collection vessel is in a sealed state. Immediately before use, the cap 301 is removed and separated from the vessel body 302. Then, a sample (e.g., a bodily fluid sample, e.g., a blood sample) is obtained from a sample source using the collection member 304, and the sample is retained in a collection channel 305 of the collection member 304. Immediately or shortly after sampling, the sealing membrane 303 of the vessel body 302 is penetrated or removed, the collection member 304 is then mounted onto the vessel body 302 to form a sealed and assembled sample collection system 300, wherein the collection channel 305 is in fluid communication with the reagents contained in the vessel body 302 and the sample collected is released from the collection channel to the vessel body, forming a reaction mixture comprising the sample and the reagents necessary for nucleic acid amplification. When the collection member 304 is mounted onto the vessel body 302, a first flange 308 comprised by the collection member 304 engages with a second flange 307 circumscribing the opening of the vessel body 302 to form a seal. In certain cases, the collection member 304 may be screw-mounted onto the vessel body 302, for example, when each of them has a matching screw thread. The assembled system 300 is then directly placed in an appropriate apparatus 306 (e.g., a PCR machine) for amplification and further analysis. The whole process may take less than about 1 hour. A point-of care amplification system of the present disclosure can provide for fast and real-time nucleic acid amplification and pathogen detection. In some embodiments, the apparatus 306 may be a heating member and may be part of the system of the present disclosure.

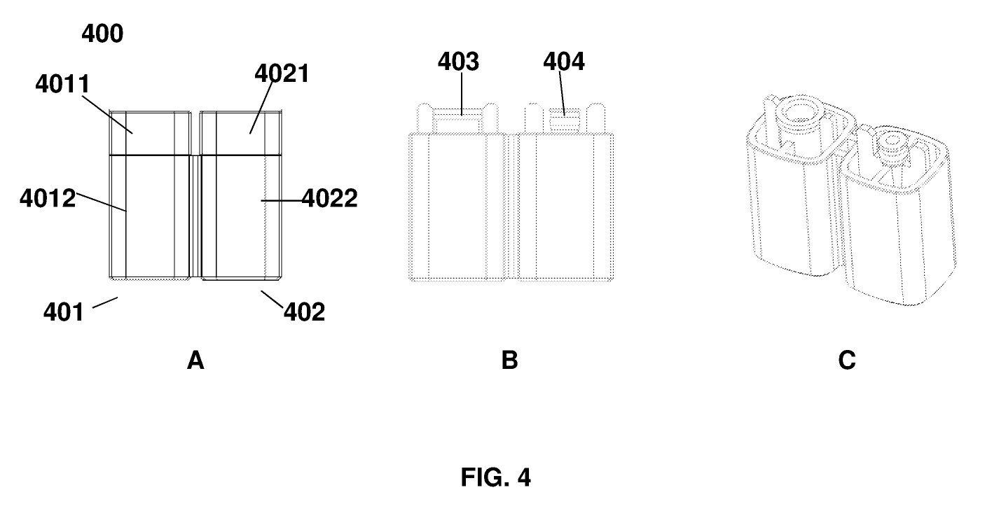

FIGS. 4A-4C provide examples of a system 400 of the present disclosure. In FIG. 4A, the system 400 comprises a first housing 401 connected to a second housing 402. The first housing 401 may comprise a housing cap 4011 and a housing body 4012, wherein the cap 4011 is removable from the housing body 4012 to expose the contents contained in the housing body 4012. The second housing 402 may comprise a housing cap 4021 and a housing body 4022, wherein the cap 4021 is removable from the housing body 4022 to expose the contents contained in the housing body 4022. FIG. 4B demonstrates the system 400 with the caps 4011 and 4021 of the housings 401 and 402 removed, respectively. In FIG. 4B, the first housing 401 may be used for storing a collection vessel 403 of the present disclosure and the second housing 402 may be used for storing a collection member 404 of the present disclosure. FIG. 4C shows a side view of the system 400 with the caps 4011 and 4021 of the housings 401 and 402 removed, respectively.

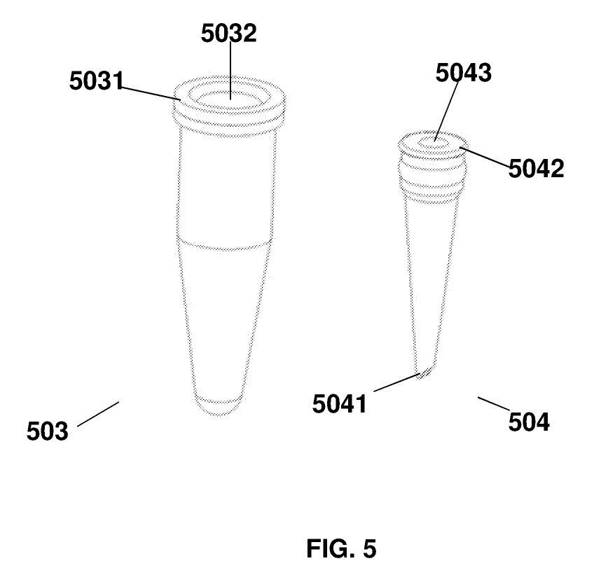

FIG. 5 provides another example of the system of the present disclosure. The system comprises a collection vessel 503 and a collection member 504. The collection member 504 may comprise a first opening 5041 at a first end of the collection member 504 and a first flange 5042 at a second end of the collection member 504. The first opening 5041 may comprise a radically extending tip that is unsymmetrical with respect to longitudinal axis of the collection member 504. The collection vessel 503 may comprise a second opening 5032 and a second flange 5031 circumscribing the second opening 5032. The collection member 504 may further comprise a third opening 5043 circumscribed by the first flange 5042. The first flange 5042 and/or second flange 5031 may be or may include a ridge, indentation and/or collar. The first flange 5042 and/or second flange 5031 may be or may include a gasket.

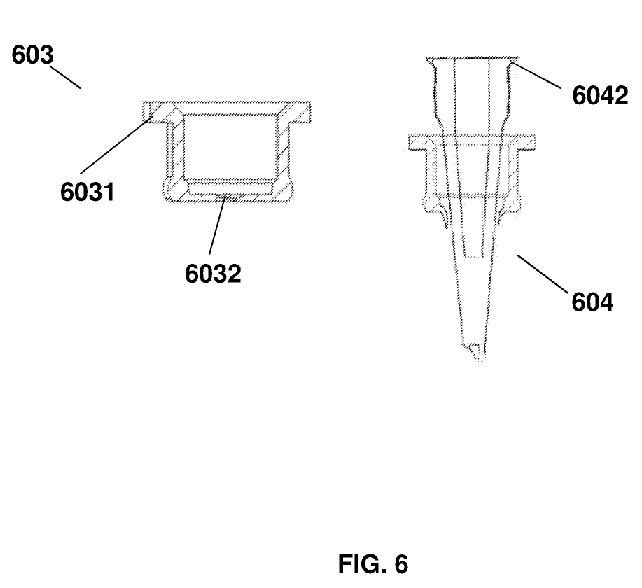

FIG. 6 shows enlarged views of part of the system of the present disclosure. The system comprises a collection vessel 603 and a collection member 604. The collection member 604 may comprise a first flange 6042. The collection vessel 603 may comprise a second flange 6031. When the collection member 604 is deposited in the collection vessel 603, the first flange 6042 engages with the second flange 6031 to form a sealed system. The collection vessel 603 may also comprise a membrane 6032 sealing the contents within the collection vessel and separate them from the surrounding environment. The membrane 6032 may have a thickness and/or strength that can be easily penetrated by the collection member 604 with the aid of a minimum amount of positive pressure. For example, the membrane 6032 may be made from parafilm, and it may have a thickness of 0.5 mm or less, such as 0.4 mm or less, 03.mm or less, 0.2 mm or less, 0.15 mm or less, 0.1 mm or less, or 0.05 mm or less.

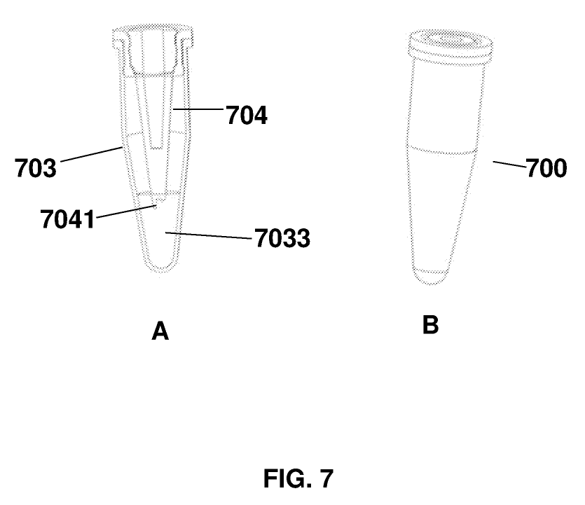

FIGS. 7A and 7B show a sectional view (FIG. 7A) and stereo view (FIG. 7B) of the system 700 after being assembled. In FIG. 7A, a collection member 704 is deposited in the collection vessel 703. The opening 7041 comprised at an end of the collection member 704 is in fluid communication with reagents 7033 comprised in the collection vessel 703. The sample (e.g., bodily fluid sample, such as a blood sample) may flow from the collection member 704 (e.g., via capillary action) into and mix with the reagents 7033 necessary for nucleic acid amplification. The assembled system 700 forms a sealed system and may be positioned in a heating member (e.g., a thermal cycler) for nucleic acid amplification.

Method for Collecting and/or Processing a Sample of a Subject

In another aspect, the present disclosure provides a method for collecting and/or processing a bodily fluid sample of a subject. First, a collection member of the present disclosure may be provided. The collection member may comprise (i) at least one collection channel in fluid communication with a first opening at a first end of the collection member, and (ii) a first flange at a second end of the collection member. Then, the first opening of the collection member may be positioned adjacent to a source of the bodily fluid sample such that the bodily fluid sample may flow from the source through the first opening to the collection channel. Next, the collection member may be deposited in a collection vessel. The collection vessel may comprise (i) a container having reagents necessary for nucleic acid amplification, and (ii) a second flange that circumscribes the second opening, wherein upon depositing the collection member in the collection vessel, the second flange engages with the first flange to form a seal. Then, the bodily fluid sample collected may flow from the collection channel through the first opening to the container (e.g., with the help of surface tension of the mixture) of the collection vessel to form a reaction mixture comprising the bodily fluid sample and the reagents. In some embodiments, when the collection member is deposited in the collection vessel, the first opening is submerged in the reagents. In some embodiments, the bodily fluid sample collected may flow from the collection channel through the first opening to the container under a positive pressure.

In some embodiments, the collection vessel may include a membrane (e.g., a polymeric membrane) adjacent to the second flange that is penetrable by the collection member. The membrane may seal the reagents within the collection vessel and separate them from the surrounding environment. The membrane may be a synthetic membrane, e.g., a membrane formed of a solid state material (e.g., semiconductor, metal, semi-metal or non-metal) or polymeric material. For example, a membrane can be formed by an opaque, transparent, or translucent material sealing a collection vessel and separating it from the external environment. Thus, in certain embodiments, when depositing the collection member in the collection vessel, the membrane may be penetrated with the collection member, rendering the collection vessel in fluid communication with the collection member, thereby permitting the sample collected to flow from the collection channel to the collection vessel. The penetrating may be achieved with the collection member of the system (e.g., comprising the sample to be analyzed). Alternatively, the penetrating may also be achieved with a different device or component that is able to penetrate the membrane.

The sample can be a tissue or a bodily fluid sample, or a fraction thereof. In some cases, the sample is a “bodily fluid” sample, which may include but not limited to blood, urine, saliva, tears, sweat, a bodily secretion, a bodily excretion, or any other fluid originating in or obtainable from a subject. In particular, the sample may include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions. For example, a sample can be a blood sample, or a portion thereof, which may include but not limited to a whole blood sample, a sample comprising red blood cells, plasma sample, serum sample, buffy coat sample, a sample comprising white blood cells, etc. The blood sample can be obtained directly from the subject, for example, the sample can be analyzed or tested (e.g., by amplification or sequencing) without further processing (e.g., by centrifugation, purification, etc.).

The nucleic acid amplification can be performed using the sample (e.g., the blood sample) deposited in the collection vessel. For example, the blood sample that is deposited in the collection vessel can be subjected to nucleic acid amplification conditions (e.g., PCR) without any additional processing of the blood sample (e.g., purification, centrifugation etc.).

The collection vessel can be substantially free of an anticoagulant. The reagents comprised in the collection vessel can include, but not limited to, one or more primers and one or more polymerizing enzymes. In certain cases, the reagents may comprise Mg or Mn ions. The reagents may further include one or more of the following: primer(s), probe(s), nucleotides (e.g., nucleotide triphosphates containing deoxyribose, or dNTP), polymerizing enzyme (or polymerase), reverse transcription enzyme (or reverse transcriptase), and/or amplification buffer. The reagents can include any one, two, three, four, five, or all of the primer(s), probe(s), nucleotides, polymerizing enzyme, reverse transcription enzyme and amplification buffer.

The one or more primers may have sequences that are selected to assay for a presence of an infectious disease in the subject. In some embodiments, the disease may be associated with a virus e.g., an RNA virus or a DNA virus. For example, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus can be selected from the group consisting of H1N1 virus, H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease may be associated with a pathogenic bacterium (e.g., Mycobacterium tuberculosis) or a pathogenic protozoan (e.g., Plasmodium).

The collection vessel can be adapted to stably store the mixture comprising the sample and the reagents for a time period of at least about 10 seconds, 30 seconds, 1 minute, 5 minutes, 10 minutes, 20 minutes, 30 minutes, 40 minutes, 50 minutes, or 60 minutes. In some embodiments, the collection vessel may be adapted to stably store the mixture for a time period of at least about 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, or 1 month.

The sample may be a bodily fluid sample having a volume of no more than about 5 mL; no more than about 4 mL; no more than about 3 mL; no more than about 2 mL; no more than about 1 mL; no more than about 500 μL; no more than about 250 μL; no more than about 100 μL; no more than about 75 μL; no more than about 50 μL; no more than about 35 μL; no more than about 25 μL; no more than about 20 μL; no more than about 15 μL; no more than about 10 μL; no more than about 8 μL; no more than about 6 μL; no more than about 5 μL; no more than about 4 μL; no more than about 3 μL; no more than about 2 μL; no more than about 1 μL; no more than about 0.8 μL; no more than about 0.5 μL; no more than about 0.3 μL; no more than about 0.2 μL; no more than about 0.1 μL; no more than about 0.05 μL; or no more than about 0.01 μL. For example, the sample can have a volume of about 0.01 μL to about 5mL, about 0.01 μL to about 4mL, about 0.01 μL to about 3mL, about 0.01 μL to about 2mL, about 0.01 μL to about 1mL, about 0.01 μL to about 0.5 μL, about 0.01 μL to about 0.4 μL, about 0.01 μL to about 0.3 μL, about 0.01 μL to about 0.2 μL, about 0.01 μL to about 0.1 μL, about 0.01 μL to about 0.05 μL.

The method may further comprise a step of disposing the collection vessel adjacent to a heating member. The heating member can be a thermal recycler for performing PCR reactions, e.g., a PCR machine. In some embodiments, the method further comprises heating the reaction mixture with the heating member. In some embodiments, the heating comprises subjecting the reaction mixture to one or more heating and cooling cycles. In some cases, the method may further comprise performing nucleic acid amplification using the reaction mixture in the collection vessel.

The heating member can be part of the system of the present disclosure. For example, the heating member can be grouped or packaged together with the collection member and/or the collection vessel, or integrated with the collection vessel, e.g., in a reversible way. In some examples, the collection vessel is integrated with the heating member, and removable from the heating member.

The method as described above or a repeating unit (e.g., a cycle) thereof may be performed in a time period of less than about 1-10 minutes. In some embodiments, the time period can be less than about 5 minutes, less than about 3 minutes, less than about 1 minute, or less than about 30 seconds.

The source of the sample can be a pool of the sample in a storage vessel. The source may also be a tissue of the subject that is accessible through a puncture in the tissue.

In some embodiments, the collection member may be stored in a first housing and the collection vessel may be stored in a second housing, the first housing and the second housing may be connected together. The connection (e.g., by physical attachment or by magnetic forces) between the first housing and the second housing may be reversibly broken. Each of said first and second housing may comprise a cap removable from a body of the housing. The housing may be used for safely storage and/or transportation of the collection member and/or the collection vessel. Before performing any analysis, the collection vessel may be in a sealed state, with reagents necessary for nucleic acid amplification comprised therein sealed by a membrane (e.g., parafilm), which could be integrated with the collection vessel.

The collection member may be sealed in a package, which may be opened immediately before use. Then, a sample (e.g., a bodily fluid sample, e.g., a blood sample) may be obtained from a sample source using the collection member, and the sample may be retained in at least one collection channel of the collection member. Immediately or shortly after sampling, the sealing membrane of the collection vessel body may be penetrated or removed with the collection member. Then, the collection member may be mounted onto the opening of the collection vessel to form a sealed and assembled sample collection system, wherein the collection channel may be in fluid communication with the reagents contained in the container of the collection vessel and the sample collected may be released from the collection channel to the container, forming a reaction mixture comprising the sample and the reagents necessary for nucleic acid amplification. When the collection member is mounted onto the collection vessel, a first flange comprised by the collection member may engage with a second flange circumscribing an opening of the collection vessel to form a seal. The assembled system may then be directly placed in a thermal cycler (e.g., a PCR machine) for amplification and further analysis. The whole process may take less than about 1 hour. A point-of care amplification system of the present disclosure can provide for fast and real-time nucleic acid amplification and pathogen detection.

Kits for Collecting and/or Processing a Bodily Fluid Sample of a Subject

Another aspect of the present disclosure provides kits for collecting and/or processing a bodily fluid sample of a subject. The kit may comprise a collection member and a collection vessel. The collection member may comprise (i) at least one collection channel in fluid communication with a first opening at a first end of the collection member, and (ii) a first flange at a second end of the collection member, wherein the first opening permits flow of the bodily fluid sample from a source of the bodily fluid sample to the collection channel. The collection vessel may comprise (i) a container having reagents necessary for nucleic acid amplification, wherein the container has a second opening at an end of the container that permits the collection member to be deposited in the container, and (ii) a second flange that circumscribes the second opening, which second flange engages with the first flange when the collection member is deposited in the to form a seal. When the collection member is deposited in the container through the second opening, the bodily fluid sample may flow from the collection channel through the first opening to the container to form a reaction mixture comprising the bodily fluid sample and the reagents.

The kit may also comprise instructions that permit a user to use the collection member to (i) collect the bodily fluid sample from the source, (ii) deposit the bodily fluid sample into the collection vessel to provide the reaction mixture and/or (iii) performing further analysis (e.g., nucleic acid amplification with the reaction mixture obtained). In some embodiments, the instructions may provide that this process is completed in a time period that is less than about 1 hour, e.g., less than about 50 minutes, less than about 40 minutes, less than about 30 minutes, less than about 20 minutes, less than about 15 minutes, less than about 10 minutes, less than about 5 minutes, less than about 4 minutes, less than about 3 minutes, less than about 2 minutes, less than about 1 minute, less than about 50 seconds, less than about 40 seconds, less than about 30 seconds, less than about 20 seconds, or less than about 10 seconds. For example, the time period can be about 10-30 seconds, in about 1-5 minutes, in about 1-10 minutes, in about 1-15 minutes, in about 1-20 minutes, in about 1-30 minutes, in about 1-40 minutes, in about 1-50 minutes or in about 1-60 minutes.

The kit may further comprise a first housing and a second housing attached to the first housing, wherein the collection member is removably stored in the first housing and the collection vessel is removably stored in the second housing. The attachment between the first housing and the second housing may be removable.

The sample can be a tissue or a bodily fluid sample, or a fraction thereof. In some cases, the sample is a “bodily fluid” sample, which may include but not limited to blood, urine, saliva, tears, sweat, a bodily secretion, a bodily excretion, or any other fluid originating in or obtainable from a subject. In particular, the sample may include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions. For example, a sample can be a blood sample, or a portion thereof, which may include but not limited to a whole blood sample, a sample comprising red blood cells, plasma sample, serum sample, buffy coat sample, a sample comprising white blood cells, etc. The blood sample can be obtained directly from the subject, for example, the sample can be analyzed or tested (e.g., by amplification or sequencing) without further processing (e.g., by centrifugation, purification, etc.).

In the kits, reagents necessary for nucleic acid amplification may include one or more primers and a polymerizing enzyme. The reagents may further include one or more of the following: primer(s), probe(s), nucleotides (e.g., nucleotide triphosphates containing deoxyribose, or dNTP), polymerizing enzyme (or polymerase), reverse transcription enzyme (or reverse transcriptase), and/or amplification buffer. The reagents can include any one, two, three, four, five, or all of the primer(s), probe(s), nucleotides, polymerizing enzyme, reverse transcription enzyme and amplification buffer. In some embodiments, the reagents include Mg or Mn ions.

The one or more primers may have sequences that are selected to assay for a presence of a disease in the subject. The disease may be an infectious disease or cancer. In some embodiments, the disease may be associated with a virus e.g., an RNA virus or a DNA virus. For example, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus can be selected from the group consisting of H1N1 virus, H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease may be associated with a pathogenic bacterium (e.g., Mycobacterium tuberculosis) or a pathogenic protozoan (e.g., Plasmodium).

In the kits, the collection vessel may be adapted to stably store the mixture for a time period of at least about 5 minutes. In some embodiments, the collection vessel may be adapted to stably store the mixture for a time period of at least about 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, or 1 month. In some embodiments, the collection channel and/or the container is substantially free of an anticoagulant.

In some embodiments, the kits may further comprise identifying information of the subject. The identifying information may be on the collection vessel, the collection member, or a housing of the collection vessel or the collection member. The identifying information may be anonymous. In some embodiments, the identifying information is on a barcode. In some embodiments, the identifying information is in a radio-frequency identification (RFID) tag.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system for collecting and/or processing a bodily fluid sample of a subject, comprising:
a collection member comprising (i) at least one collection channel in fluid communication with a first opening at a first end of said collection member, and (ii) a first flange at a second end of said collection member, wherein said first opening permits flow of said bodily fluid sample from a source of said bodily fluid sample to said collection channel; and
a collection vessel comprising (i) a container having reagents necessary for nucleic acid amplification, wherein said container has a second opening at an end of said container that permits said collection member to be deposited in said container, and (ii) a second flange that circumscribes said second opening, which second flange engages with said first flange when said collection member is deposited in said container to form a seal,
wherein when said collection member is deposited in said container through said second opening, said bodily fluid sample flows from said collection channel through said first opening to said container to form a reaction mixture comprising said bodily fluid sample and said reagents.

2. The system of claim 1, wherein said first opening and/or said collection channel is dimensioned to permit flow of said bodily fluid sample via capillary action.

3. The system of claim 1, wherein said collection member further comprises a container in fluid communication with said collection channel, wherein said container has a larger cross-sectional area than said collection channel.

4. The system of claim 1, wherein said collection member further comprises a tip.

5. The system of claim 4, wherein said tip includes a needle.

6. The system of claim 4, wherein said tip is a radially extending tip that is asymmetrical with respect to longitudinal axis of said collection member.

7. The system of claim 1, wherein said collection member comprises a third opening that is dimensioned to seal said collection channel upon contact of said first flange with an external object.

8. The system of claim 7, wherein said first flange circumscribes said third opening.

9. (canceled)

10. The system of claim 1, wherein said collection member is removably stored in a first housing and said collection vessel is removably stored in a second housing that is attached to said first housing.

11. The system of claim 1, wherein said collection vessel includes a polymeric membrane adjacent to said second flange.

12. The system of claim 11, wherein said polymeric membrane is sealable or resealable.

13.-15. (canceled)

16. The system of claim 1, wherein said collection channel and/or said container is substantially free of an anticoagulant.

17. (canceled)

18. The system of claim 1, wherein said reagents comprise one or more primers having sequences that are configured to assay for a presence of a disease in said subject.

19. (canceled)

20. The system of claim 1, wherein said reagents include magnesium Mg or manganese Mn ions.

21. The system of claim 1, wherein said collection vessel is adapted to stably store said reaction mixture for a time period of at least about 5 minutes.

22.-25. (canceled)

26. The system of claim 1, wherein said collection vessel, said collection member, or a housing associated with said collection vessel or said collection member comprises identifying information of said subject.

27. (canceled)

28. The system of claim 25, wherein said identifying information is encoded in a barcode.

29. The system of claim 25, wherein said identifying information is encoded in a radio-frequency identification (RFID) tag.

30. The system of claim 1, further comprising a heating member adjacent to said collection vessel, wherein said heating member heats said reaction mixture during nucleic acid amplification.

31. The system of claim 30, wherein said heating member is a thermal cycler that subjects said reaction mixture to one or more heating and cooling cycles during said nucleic acid amplification.

32. The system of claim 30, wherein said heating member comprises a receptacle that is dimensioned to accept said collection vessel.

33. The system of claim 1, wherein said collection member comprises a plurality of collection channels.

34. A method for collecting and/or processing a bodily fluid sample of a subject, comprising:
(a) providing a collection member comprising (i) at least one collection channel in fluid communication with a first opening at a first end of said collection member, and (ii) a first flange at a second end of said collection member;
(b) positioning said first opening of said collection member adjacent to a source of said bodily fluid sample such that said bodily fluid sample flows from said source through said first opening to said collection channel;
(c) depositing said collection member in a collection vessel comprising (i) a container having reagents necessary for nucleic acid amplification, and (ii) a second flange that circumscribes said second opening, wherein upon depositing said collection member in said collection vessel, said second flange engages with said first flange to form a seal; and
(d) flowing said bodily fluid sample from said collection channel through said first opening to said container to form a reaction mixture comprising said bodily fluid sample and said reagents.

35.-52. (canceled)

53. A kit for collecting and/or processing a bodily fluid sample of a subject, comprising:
a collection member comprising (i) at least one collection channel in fluid communication with a first opening at a first end of said collection member, and (ii) a first flange at a second end of said collection member, wherein said first opening permits flow of said bodily fluid sample from a source of said bodily fluid sample to said collection channel;
a collection vessel comprising (i) a container having reagents necessary for nucleic acid amplification, wherein said container has a second opening at an end of said container that permits said collection member to be deposited in said container, and (ii) a second flange that circumscribes said second opening, which second flange engages with said first flange when said collection member is deposited in said to form a seal, wherein when said collection member is deposited in said container through said second opening, said bodily fluid sample flows from said collection channel through said first opening to said container to form a reaction mixture comprising said bodily fluid sample and said reagents; and
instructions that permit a user to use said collection member to (i) collect said bodily fluid sample from said source, and (ii) deposit said bodily fluid sample into said collection vessel to provide said reaction mixture.

54.-64. (canceled)
